(19) United States
(12) Patent Application Publication
(10) Pub. No.: US 2012/0225126 A1
(43) Pub. Date: Sep. 6, 2012

(54) SOLID STATE SYNTHESIS METHOD OF SILVER NANOPARTICLES, AND SILVER NANOPARTICLES SYNTHESIZED THEREBY

(75) Inventors: Kurt E. Geckeler, Buk-gu (KR); Dipen Debnath, Buk-gu (KR); Chorong Kim, Buk-gu (KR); Sung Ho Kim, Buk-gu (KR)

(73) Assignee: GWANGJU INSTITUTE OF SCIENCE AND TECHNOLOGY, Buk-gu, Gwangju (KR)

(21) Appl. No.: 13/509,463

(22) PCT Filed: Nov. 9, 2010
(86) PCT No.: PCT/KR2010/007887

§ 371 Date: May 11, 2012

(30) Foreign Application Priority Data
Nov. 11, 2009 (KR) 10-2009-0108405

(51) Int. Cl. A01N 25/26; A01N 55/02; A01P 1/00; A01N 59/16; B82Y 5/00

(52) U.S. Cl. 424/491; 424/490; 424/493; 424/494; 424/618; 424/619; 514/495; 977/773; 977/904

(57) ABSTRACT

Disclosed are a solid state synthesis method of silver nanoparticles, and silver nanoparticles synthesized thereby. The method includes mixing a silver salt and a water soluble polymer acting as both a reducing agent and a protecting agent to produce a solid mixture, and milling the solid mixture by a high-speed vibration milling process to form silver nanoparticles within the water soluble polymer. According to the present invention, silver nanoparticles can be easily and simply produced in a solid state through high speed vibration milling, thereby reducing costs for industrial production and transportation of silver nanoparticles. In addition, the synthesized silver nanoparticles can be used for a long time since the silver nanoparticles are stable in a solid state for 1 year or more.

### TECHNICAL FIELD

The present invention relates to a synthesis method of silver nanoparticles, and silver nanoparticles synthesized thereby, and more particularly, to a method of synthesizing silver nanoparticles by solid state reaction and silver nanoparticles synthesized thereby.

### BACKGROUND ART

Recently, nanoparticles (NPs) have attracted attention due to their unique electrical, optical, magnetic and photoelectric properties, and applicability based on such properties in various fields of electrical engineering, medical science, biotechnology, environmental science, energy, and the like. Among metal nanoparticles, silver nanoparticles (AgNPs) are very industrially applicable, since silver is a noble metal and 70% or more of silver produced in the world is used for industrial purposes.

Currently, although various synthesis methods of silver nanoparticles have been reported, most conventional methods are carried out in a liquid state. Liquid state synthesis is advantageous in forming uniform nanoparticles through selective synthesis of nanoparticles having a certain particle size and separation of the synthesized nanoparticles by adjusting various synthesis conditions. However, colloidal particles tend to agglomerate in a liquid phase due to their high surface energy, and a protecting agent such as polymers, surfactants or thiols is generally used to prevent such agglomeration of the colloidal particles by surrounding and stabilizing the particles. Further, such a protecting agent provides an important function in adjusting the size and shape of the nanoparticles.

However, the liquid-state synthesis method is not suited to mass production of commercial silver nanoparticles at low cost. In order to guarantee dispersion stability in the liquid-state synthesis method, it is necessary to maintain a very low concentration of metal in a liquid state. Accordingly, a large amount of dispersion media is necessarily used together with a very large container for mass production and transportation of silver nanoparticles, causing an increase in manufacturing cost. Moreover, morphology change of the synthesized silver nanoparticles can occur during a process of evaporating a solvent in preparation of a solid sample from a liquid phase. As such, the conventional liquid-state synthesis method of silver nanoparticles is not suitable in terms of commercial mass production. Therefore, there is still a need for a method of mass producing silver nanoparticles at low cost.

### DISCLOSURE

### Technical Problem

The present invention is aimed at providing a method of synthesizing silver nanoparticles by solid state reaction and silver nanoparticles synthesized thereby.

### Technical Solution

An aspect of the present invention provides a solid state synthesis method of silver nanoparticles. The method includes: mixing a silver salt and a water soluble polymer acting as both a reducing agent and a protecting agent to produce a solid mixture; and milling the solid mixture by a high-speed vibration milling process to form silver nanoparticles within the water soluble polymer.

The silver salt may be one selected from the group consisting of silver nitrate (AgNO₃), silver nitrite (AgNO₂), silver acetate (CH₃COOAg), silver lactate (CH₃CH(OH)COOAg), silver citrate hydrate (AgO₂CCH₂C(OH)(CO₂Ag)CH₂CO₂Ag.xH₂O), and mixtures thereof.

The water soluble polymer may include oxygen or nitrogen having lone pair electrons.

The water soluble polymer including the oxygen or nitrogen having lone pair electrons may be one selected from the group consisting of starch, amylopectin, amylose, cellulose, cellulose acetate, nitrocellulose, ethyl cellulose, hydroxypropyl cellulose, sodium carboxymethyl cellulose, chitin, chitosan, glycogen, poly(acrylic acid), poly(L-alanine), poly(ethylene glycol), polyglycine, poly(glycolic acid), poly(2-hydroxyethyl methacrylate), poly(vinyl pyrrolidone), and mixtures thereof.

Another aspect of the present invention provides silver nanoparticles. The silver nanoparticles may be prepared by the solid state synthesis method.

The prepared silver nanoparticles may have an average particle diameter ranging from 2 to 50 nm.

### Advantageous Effects

According to the present invention, the synthesis method may easily and conveniently produce silver nanoparticles from a solid phase through a high-speed vibration milling process. Namely, the synthesis method does not need any solvent for synthesis and transportation of silver nanoparticles and may eliminate a large container for silver nanoparticles. In addition, the synthesis method may produce silver nanoparticles from a silver nanoparticle precursor without a separate reducing agent. As a result, the synthesis method according to the present invention may reduce costs for production and transportation of silver nanoparticles.

In addition, silver nanoparticles synthesized by the method according to the present invention are stable in solid state for 1 year or more, thereby enabling use of the prepared silver nanoparticles for a long period of time. In particular, the silver nanoparticles synthesized by the method may be used as a strong antimicrobial agent.

It will be understood by those skilled in the art that the present invention is not limited to these effects and other effects will become apparent from the following description.

### DESCRIPTION OF DRAWINGS

FIG. 1 is a schematic view illustrating a solid state synthesis method of silver nanoparticles according to one exemplary embodiment of the present invention.

FIG. 2 shows UV-Vis absorption spectra of dispersion liquids A to C.

FIGS. 3 to 5 are TEM images of samples A to C.

FIGS. 6 to 8 are histograms of particle size distribution shown in the TEM images of FIGS. 3 to 5, respectively.

FIG. 9 to FIG. 11 are high resolution TEM images of samples A to C, in which an image at an upper right side is an FFT image of a region denoted by a rectangular solid line.

FIG. 12 FT-IR spectra of sample A, sample B, and PVP not containing silver nanoparticles.

FIG. 13 is an image of an antimicrobial property test result with respect to silver nanoparticles prepared according to one example of the present invention.

FIG. 14 is a graph depicting size variation of a bacteria growth inhibitory zone according to time.

### BEST MODE

Exemplary embodiments of the present invention will now be described in more detail with reference to the accompanying drawings. It should be understood that the present invention is not limited to the following embodiments and may be embodied in different ways, and that the embodiments are given to provide complete disclosure of the invention and to provide a thorough understanding of the invention to those skilled in the art. Description of details apparent to those skilled in the art will be omitted for clarity.

In accordance with one aspect, the present invention provides a solid state synthesis method of silver nanoparticles, which includes: mixing a silver salt and a water soluble polymer acting as both a reducing agent and a protecting agent to produce a solid mixture; and milling the solid mixture by a high-speed vibration milling process to form silver nanoparticles within the water soluble polymer.

The silver salt acts as a silver nanoparticle precursor, which forms silver nanoparticles through reduction and agglomeration of a silver core. The silver salt may be one selected from the group consisting of silver nitrate (AgNO₃), silver nitrite (AgNO₂), silver acetate (CH₃COOAg), silver lactate (CH₃CH(OH)COOAg), silver citrate hydrate (AgO₂CCH₂C(OH)(CO₂Ag)CH₂CO₂Ag.xH₂O), and mixtures thereof.

In synthesis of the silver nanoparticles, the water soluble polymer acts as both a reducing agent for the silver nanoparticle precursor (specifically, silver cations, Ag⁺) and a protecting agent for the synthesized silver nanoparticles. Advantageously, the water soluble polymer includes oxygen or nitrogen having lone pair electrons. Here, the lone pair electrons provide driving force of promoting interaction between the water soluble polymer and silver particles (including the silver cations and the silver nanoparticles), and allow the water soluble polymer to act as both a reducing agent and a protecting agent. The water soluble polymer including the oxygen or nitrogen having lone pair electrons may be one selected from the group consisting of starch, amylopectin, amylose, cellulose, cellulose acetate, nitrocellulose, ethyl cellulose, hydroxypropyl cellulose, sodium carboxymethyl cellulose, chitin, chitosan, glycogen, poly(acrylic acid), poly(L-alanine), poly(ethylene glycol), polyglycine, poly(glycolic acid), poly(2-hydroxyethyl methacrylate), poly(vinyl pyrrolidone), and mixtures thereof

In accordance with another aspect, the present invention provides silver nanoparticles produced by the solid state synthesis method as described above. The silver nanoparticles produced by the solid state synthesis method may have a particle size ranging from 2 to 50 nm through suitable adjustment of the kinds and amounts of the silver nanoparticle precursor and water soluble polymer.

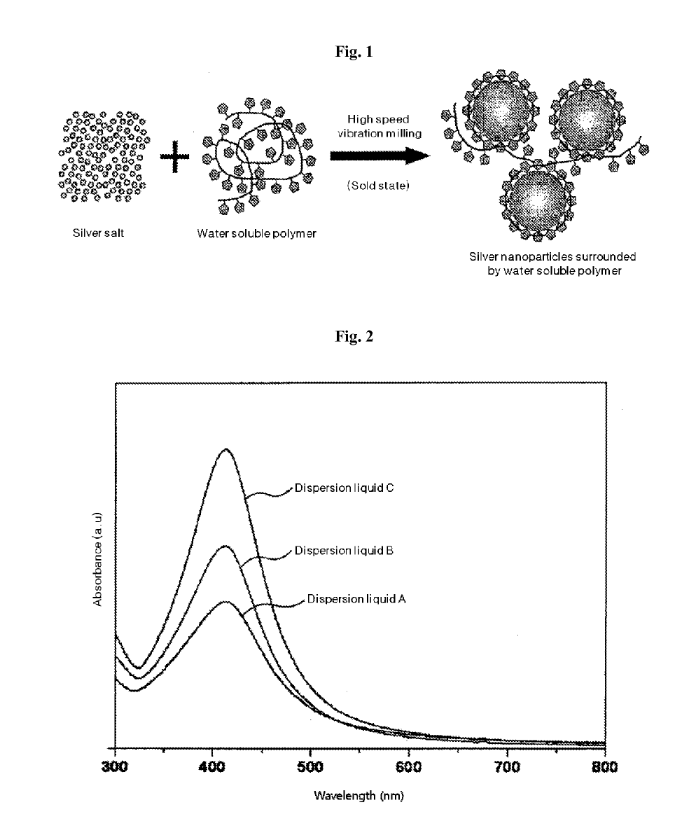

FIG. 1 is a schematic view illustrating a solid state synthesis method of silver nanoparticles according to one exemplary embodiment of the present invention, in which silver nitrate is used as the silver salt and poly(vinyl pyrrolidone) is used as the water soluble polymer.

As shown in FIG. 1, in this embodiment, the silver salt and the water soluble polymer are mixed to prepare a solid-state mixture, which in turn is subjected to a high-speed vibration milling process, thereby producing silver nanoparticles surrounded by the water soluble polymer.

The synthesis mechanism of silver nanoparticles has not been clearly elucidated. However, it can be recognized that the synthesis mechanism of the silver nanoparticles through high-speed vibration milling in a solid state results from thermodynamic control and is related to a series of processes as follows. In the process of synthesizing the silver nanoparticles, the water soluble polymer acts not only as a reducing agent of silver cations by forming a complex compound, but also as a protecting agent of the silver nanoparticles, and it is recognized that such functions of the water soluble polymer are closely related to interaction between the water soluble polymer and surfaces of the silver particles. In other words, during high-speed vibration milling, the complex compound of the silver cations and the water soluble polymer is formed first, and reduction of the silver cations occurs by the unpaired electrons of the water soluble polymer. After reduction of the silver cations, the water soluble polymer is detached from the surface of silver atoms, followed by agglomeration of the silver core through rearrangement of the silver atoms, thereby forming silver nanoparticles. Here, the unpaired electrons of the water soluble polymer provided to the silver cations may be supplemented by counter-anions of the silver cations. Finally, the surfaces of the silver nanoparticles are protected by formation of the complex compound of the silver nanoparticles and the water soluble polymer.

Next, the present invention will be explained in more detail with reference to examples. It will be apparent to those skilled in the art that these examples are provided for illustrative purposes only and are not to be in any way construed as limiting the present invention.

<Synthesis of Silver Nanoparticles through Solid-State Reaction Process>

### Preparation Example 1

15 mg (0.08 mmol) of silver nitrate (AgNO₃) and 100 mg (0.9 mmol) of poly(vinyl pyrrolidone) (PVP, Mw=30 kg/mol) (weight ratio=1.5:10) were placed together with an agate mixing ball in an agate capsule. Then, the mixture was intensely mixed at 1500 rpm and ambient temperature for 15 minutes using a high speed vibration mill (MM 200, Retsch Co., Ltd.). As a result, silver nanoparticles surround by the water soluble polymer were obtained. The resultants (“sample A”) were a solid mixture and had deep yellow to yellow colors.

### Preparation Example 2

Silver nanoparticles (“sample B”) surrounded by the water soluble polymer were obtained by the same method as in Preparation Example 1 except that the silver nitrate and PVP were mixed in a weight ratio of 3:10.

### Preparation Example 3

Silver nanoparticles (“sample C”) surrounded by the water soluble polymer were obtained by the same method as in Preparation Example 1 except that the silver nitrate and PVP were mixed in a weight ratio of 5:10.

<Property Analysis of Silver Nanoparticles>

To observe the properties of the silver nanoparticles, samples A to C were dispersed in water (5 mg/ml) to prepare a silver nanoparticle dispersion liquid in a colloidal state. Hereinafter, the dispersion liquids containing samples A, B and C will be referred to as dispersion liquids A, B and C, respectively.

Analysis of Optical Characteristics

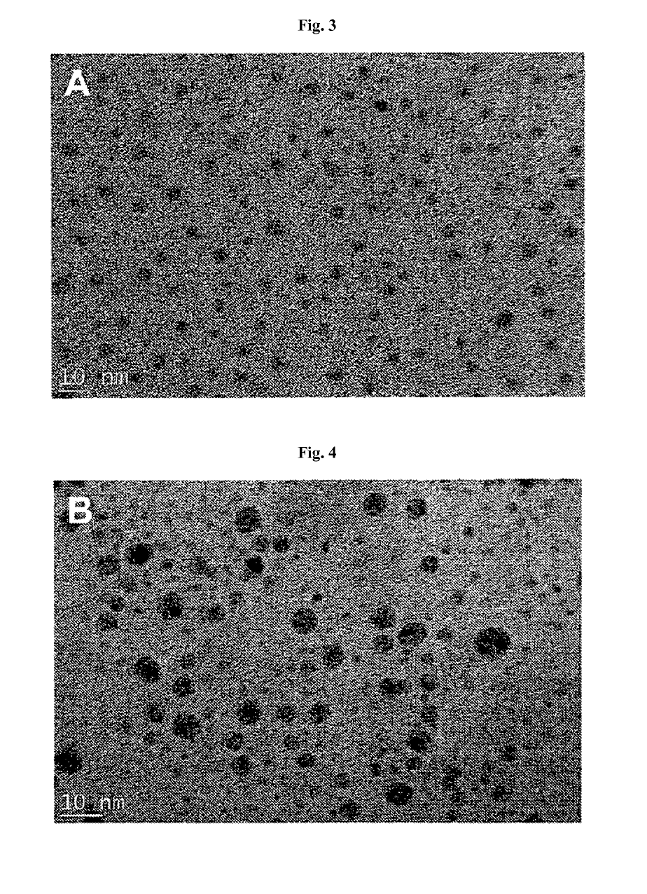

FIG. 2 shows UV-Vis absorption spectra of dispersion liquids A to C.

In FIG. 2, the UV-Vis absorption spectra were obtained by placing the dispersion liquids in 1 cm×1 cm×3 cm UV cuvettes, followed by measuring at room temperature at a resolution of 1 nm at wavelengths of 300 to 800 nm using a Carry 1E UV-Vis spectrophotometer (Varian 95011211).

As clearly shown in FIG. 2, all of absorption bands of the respective dispersion liquids showed maximum absorbance near a wavelength of about 412 nm, corresponding to a typical absorption band of silver nanoparticles. As a result, it could be seen that the silver nanoparticles were produced by the solid state synthesis method according to the present invention.

Analysis of Size and Crystallinity of Silver Nanoparticles

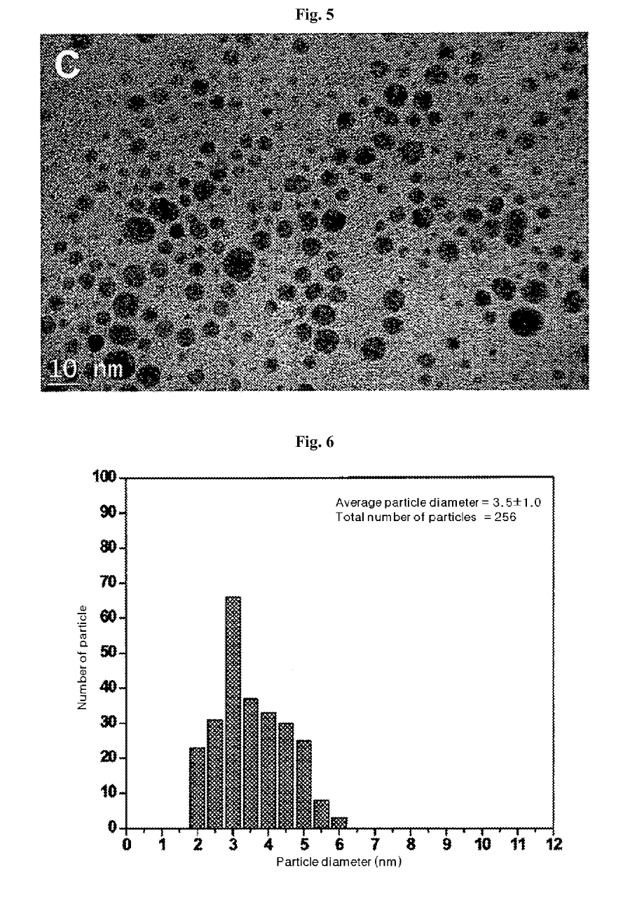

FIGS. 3 to 5 are TEM images of samples A to C.

In FIGS. 3 to 5, the TEM images were obtained using a transmission electron microscope (JEOL JEM-2100) at 200 kV. The samples for TEM analysis were obtained by dropping each of dispersion liquids A to C on a carbon-coated copper grid, followed by drying in air.

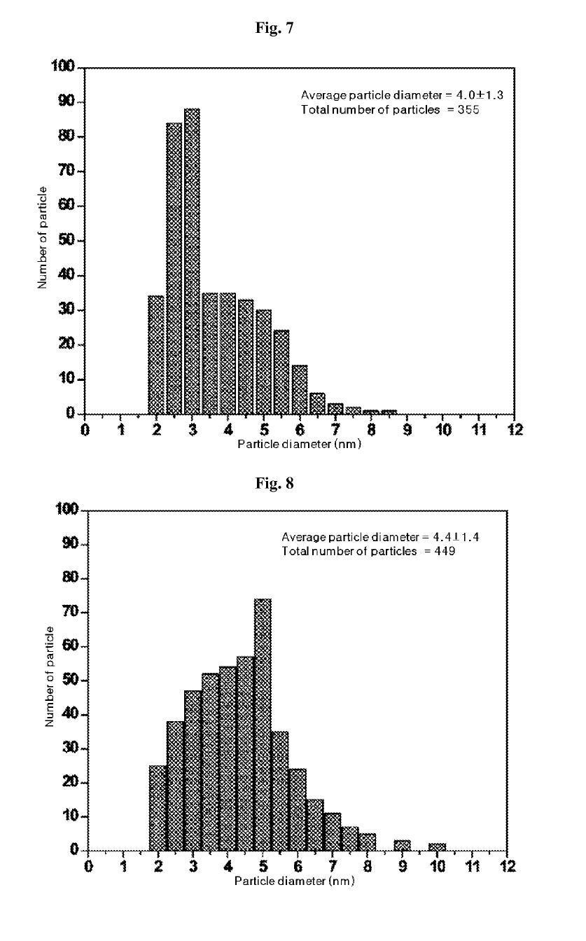

FIGS. 6 to 8 are histograms of particle size distribution shown in the TEM images of FIGS. 3 to 5, respectively.

Referring to FIGS. 3 to 8, it could be seen that the average particle size and the number of prepared silver nanoparticles increased with increasing amount of the mixed silver precursor (silver nitrate). Here, it could also be seen that the average particle size of the silver nanoparticles increased narrowly due to an increase in the number of particles. Samples A, B and C had average particle sizes of 3.5±1.0 nm, 4.0±1.3 nm, and 4.4±1.4 nm, respectively.

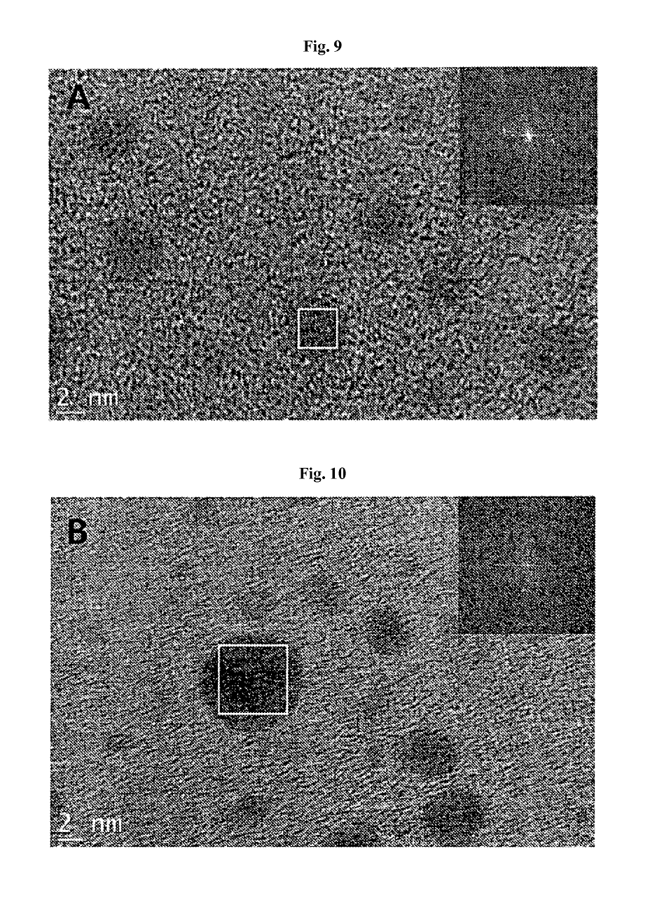

FIGS. 9 to 11 are high resolution TEM images of samples A to C, in which an image at an upper right side is an FFT image of a region denoted by a rectangular solid line.

As shown in FIGS. 9 to 11, it could be seen through high resolution TEM images and Fast Fourier Transform (FFT) analysis that the silver nanoparticles of samples A and B were single crystals. Conversely, the silver nanoparticles of sample C were large crystals composed of many crystalline particles rather than single crystals, and it could be confirmed that small particles forming the large particles were single crystals. Since sample C had a smaller concentration of the mixed protecting agent (water soluble polymer) than samples A and B, it was more or less difficult to achieve effective stabilization of single nanoparticles without additional agglomeration. That is, for sample C, it was recognized that the polycrystalline nanoparticles were formed by additional agglomeration of the small single crystalline nanoparticles, which were not suitably stabilized. However, for samples A and B, it was recognized that, since the amount of the mixed protecting agent was sufficiently high to stabilize the synthesized single crystal nanoparticles, it was possible to avoid additional agglomeration of the single crystalline nanoparticles so as not to form polycrystalline nanoparticles during the reaction.

It can be seen from analysis of FIG. 3 to FIG. 11 that crystallinity and size of the nanoparticles synthesized by the method according to the present invention depends on the amount of the silver precursor.

Analysis of Synthesis Mechanism of Silver Nanoparticles

Synthesis mechanism of silver nanoparticles was observed using Fourier Transform-Infrared (FT-IR) spectroscopy.

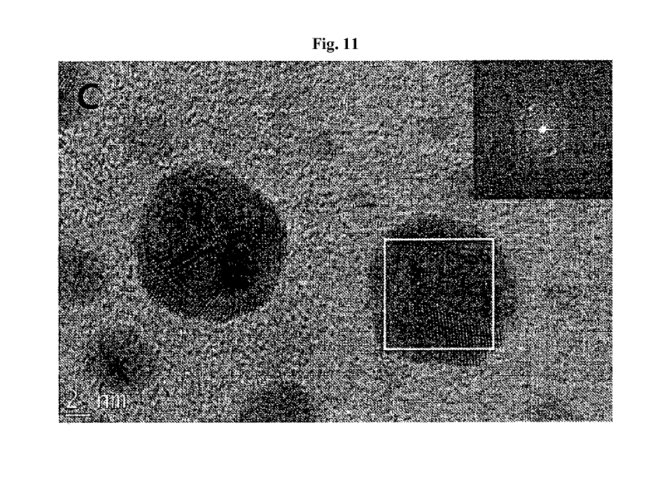

FIG. 12 shows FT-IR spectra of sample A, sample B, and PVP not containing silver nanoparticles (“pure PVP”).

In FIG. 12, the FT-IR spectra were obtained using a Perkin-Elmer FT-IR spectrometer 2000 and samples prepared using KBr pellets.

As shown in FIG. 12, the carbonyl (C═O) absorption band of PVP included in samples A and B did not show a considerable shift as compared with the carbonyl absorption band (1667 cm⁻¹) of the pure PVP. This result means that oxygen of the carbonyl group did not participate in reduction of the silver cations (Ag+) and stabilization of the synthesized silver nanoparticles. However, as compared with the pure PVP, samples A and B exhibited new absorption bands with respect to C—N not only at 1018 cm⁻¹ but also at 1034 cm⁻¹, and this result means that electrons of pyrrolidyl nitrogen participated in formation of the silver nanoparticles. In other words, pyrrolidyl nitrogen participated in reduction of the silver cations (Ag+) and stabilization of the synthesized silver nanoparticles instead of oxygen of the carbonyl group, irrespective of steric hindrance with respect to electron donating from N to Ag⁺ and coordination between N and Ag⁺. As a reason for this result, it is recognized that the lower electron negativity of nitrogen as compared to oxygen overcomes steric hindrance to provide driving force of promoting participation of nitrogen atoms in formation of the silver nanoparticles. Accordingly, in a C—N bond including nitrogen participating in formation of the silver nanoparticles, the C—N vibration frequency is shifted from 1018 cm⁻¹ to 1034 cm⁻¹. However, in the case where the nitrogen atoms do not participate in formation of the silver nanoparticles, the C—N vibration frequency is not shifted, and the absorption bands are observed at two regions. Therefore, it could be seen from the red-shift of the C—N vibration frequencies based on the IR spectroscopic analysis that pyrrolidyl nitrogen (specifically, unpaired electrons of pyrrolidyl nitrogen) served to reduce silver ions into silver metal and to stabilize the synthesized silver nanoparticles.

Analysis of Antimicrobial Properties

Antimicrobial activity of the prepared silver nanoparticles was evaluated by an in vitro disk diffusion test of a Kirby-Bauer process. Escherichia coli KCTC 1682 of Gram negative bacteria was obtained from the Korean Collection for Type Cultures (KCTC). The bacteria were placed in a pattern of diagonal lines on a Mueller Hinton agar plate by streaking thereon using a wire loop, and then incubated at 37 for 18 hours. 5 ml of MH broth nutrient solution was injected into a single colony selected from the agar plate and exhibiting the same shape, followed by incubation at 37 for 5 hours to match 0.5 McFarland standard (absorbance at a wavelength of 625 nm=0.096,about 10⁸ organisms/ml). Then, the incubated bacteria were removed from the culture and then placed on a sufficiently dried agar surface of another growth culture.

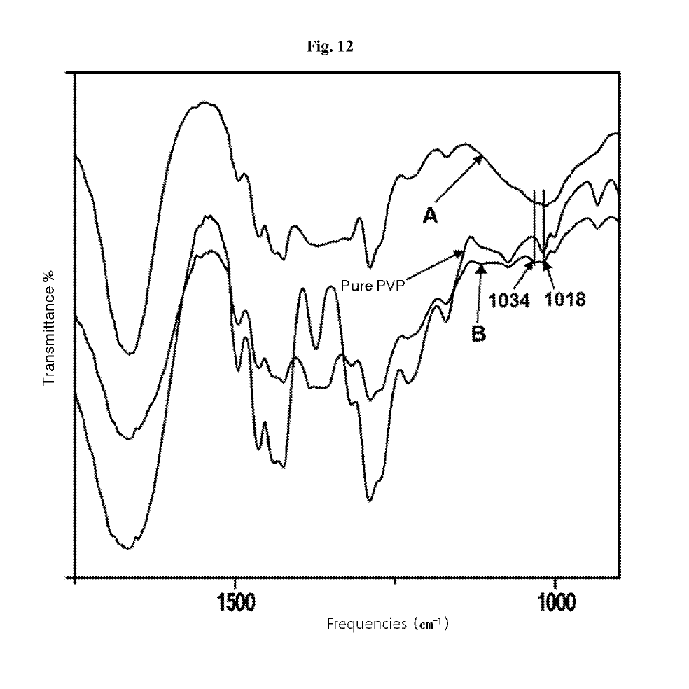

Three disks, each of which absorbed 100 μl of sample A, B or C (each containing 0.68 ppm of silver) (A, B and C of FIG. 13), were dried in an oven. In addition, a pure PVP-absorbing disk (P of FIG. 13) and a water-absorbing disk (W of FIG. 13) were also prepared. The prepared 5 disks were placed in the 100 mm plate culture inoculated with the bacteria, followed by incubation at 37 for 2 hours and measurement of the growth inhibitory zones each 2 hours for 24 hours. In this testing, all of the processes were performed according to the procedures of the National Committee for Clinical Laboratory Standards (NCCLS) in order to provide reliable results.

FIG. 13 is an image of an antimicrobial property test result with respect to silver nanoparticles prepared according to one example of the present invention.

Referring to FIG. 13, the bacteria (E. Coli) growth inhibitory zones having sizes (diameters) of 0.95 cm, 0.9 cm and 0.9 cm were respectively formed around the silver nanoparticle containing disks A, B and C, and the antimicrobial properties of the silver nanoparticles were ascertained.

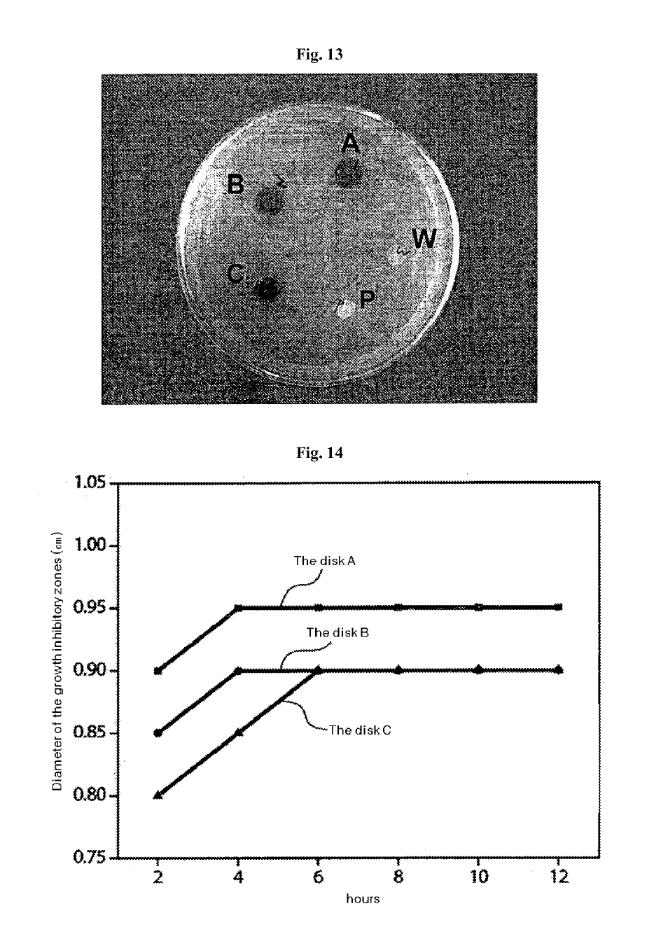

FIG. 14 is a graph depicting size variation of a bacteria growth inhibitory zone according to time.

As shown in FIG. 14, for the disks A and B, the growth inhibitory zones had the greatest size after 4 hours, whereas the disk C had the greatest growth inhibitory zone after 6 hours. It was recognized that this result was caused by rapid antimicrobial activity due to a larger specific surface area of the silver nanoparticles contained in the disks A and B than those of the disk C.

Although some exemplary embodiments have been described with reference to the accompanying drawing, it will be understood by those skilled in the art that these embodiments are provided by way of illustration only and do not limit the scope of the present invention. Therefore, the scope and sprit of the present invention should be defined by the accompanying claims and equivalents thereof.

1. A solid state synthesis method of silver nanoparticles, comprising:
mixing a silver salt and a water soluble polymer acting as both a reducing agent and a protecting agent to produce a solid mixture; and
milling the solid mixture by a high-speed vibration milling process to form silver nanoparticles within the water soluble polymer.

2. The solid state synthesis method according to claim 1, wherein the silver salt is one selected from the group consisting of silver nitrate (AgNO₃), silver nitrite (AgNO₂), silver acetate (CH₃COOAg), silver lactate (CH₃CH(OH)COOAg), silver citrate hydrate (AgO₂CCH₂C(OH)(CO₂Ag)CH₂CO₂Ag.xH₂O), and mixtures thereof.

3. The solid state synthesis method according to claim 1, wherein the water soluble polymer comprises oxygen or nitrogen having lone pair electrons.

4. The solid state synthesis method according to claim 3, wherein the water soluble polymer comprising the oxygen or nitrogen having lone pair electrons is one selected from the group consisting of starch, amylopectin, amylose, cellulose, cellulose acetate, nitrocellulose, ethyl cellulose, hydroxypropyl cellulose, sodium carboxymethyl cellulose, chitin, chitosan, glycogen, poly(acrylic acid), poly(L-alanine), poly(ethylene glycol), polyglycine, poly(glycolic acid), poly(2-hydroxyethyl methacrylate),

5. Silver nanoparticles prepared by the solid state synthesis method according to claim 1.

6. Silver nanoparticles prepared by the solid state synthesis method according to claim 2.

7. Silver nanoparticles prepared by the solid state synthesis method according to claim 3.

8. Silver nanoparticles prepared by the solid state synthesis method according to claim 4.

9. The silver nanoparticles according to claim 5, wherein the silver nanoparticles have an average particle diameter ranging from 2 to 50 nm.

10. The silver nanoparticles according to claim 6, wherein the silver nanoparticles have an average particle diameter ranging from 2 to 50 nm.

11. The silver nanoparticles according to claim 7, wherein the silver nanoparticles have an average particle diameter ranging from 2 to 50 nm.

12. The silver nanoparticles according to claim 8, wherein the silver nanoparticles have an average particle diameter ranging from 2 to 50 nm.
